(19) United States
(12) Patent Application Publication
(10) Pub. No.: US 2023/0173053 A1
(43) Pub. Date: Jun. 8, 2023

(54) Lyophilized Live Bordetella Vaccines

(71) Applicant: ILiAD Biotechnologies, LLC, Weston, FL (US)

(72) Inventor: Marcel Thalen, Frasnes lez Anvaing (BE)

(21) Appl. No.: 18/159,550

(22) Filed: Jan. 25, 2023

Related U.S. Application Data
(63) Continuation-in-part of application No. PCT/US2021/046055, filed on Aug. 15, 2021.
(60) Provisional application No. 63/066,020, filed on Aug. 14, 2020.

(51) Int. Cl. A61K 39/02; A61K 47/26; A61K 47/18; A61K 9/19; C12N 1/04; C12N 1/20

(52) U.S. Cl. CPC A61K 39/099; A61K 9/19; A61K 47/26; A61K 47/183; C12N 1/04; C12N 1/20; A61K 2039/522

(57) ABSTRACT

Formulations of lyophilized Bordetella bacteria which are stable for at least two years when stored at temperatures between -20° and 22.5° C., and which exhibit sufficient homogeneity (no bacterial clumping) bacterial viability and potency to be used as a live vaccine are made by harvesting Bordetella bacteria from a culture at an OD600 between 0.4 and 1.6; mixing the harvested Bordetella bacteria with a lyophilization buffer comprising 5-65% by weight a cryoprotectant sugar and having a temperature between 2-3 5° C., wherein the ratio of harvested Bordetella bacteria to lyophilization buffer is between 5:1 and 1:5 by volume; lyophilizing the mixture of the Bordetella bacteria and the lyophilization buffer; wherein the hold time between the harvesting and lyophilization steps is less than 48 hours; and collecting the lyophilized Bordetella bacteria.

### CROSS-REFERENCE TO RELATED APPLICATIONS

The present application is a by-pass continuation-in-part under 35 U.S.C. 111(a) of International Pat. Application No. PCT/US2021/046055 filed on Aug. 15, 2021 which claims the priority of U.S. Provisional Pat. Application Serial No. 63/066,020 filed on Aug. 14, 2020.

### STATEMENT AS TO FEDERALLY SPONSORED RESEARCH

Not applicable.

### FIELD OF THE INVENTION

The invention relates generally to the fields of microbiology, vaccines, and lyophilization, and more specifically to methods for growing and lyophilizing Bordetella bacteria and lyophilized formulations made according to such methods.

### BACKGROUND

BPZE1, a live attenuated B. pertussis strain, was previously developed for use in a whooping cough (pertussis) vaccine. See U.S. Pat. No. 9,180,178. This vaccine strain was constructed by genetically removing dermonecrotic toxin, reducing tracheal cytotoxin to background levels, and inactivating pertussis toxin. In a non-human primate model, a single nasal administration of BPZE1 was found to provide strong protection against both pertussis disease and infection, following a challenge by a highly virulent recent clinical B. pertussis isolate. BPZE1 is now in clinical development and has already successfully completed two phase I studies, which have shown that the vaccine is safe in adult volunteers, able to transiently colonize the human nasal cavity and to induce antibody responses to B. pertussis antigens. The liquid formulation of BPZE1 used in these previous studies requires storage at -70° C. to maintain bacterial viability. Because most point-of-care facilities are not equipped with ultra low freezers, this requirement is an impediment to the future commercialization of BPZE1-based vaccines.

### SUMMARY

Described herein are formulations and methods of making formulations of lyophilized Bordetella bacteria which are stable for at least two (e.g., 2, 3, or 3.5) years when stored at temperatures between -20° and 22.5° C., and which exhibit sufficient homogeneity (no clumping), bacterial viability and potency to be used as a live vaccine. Prior to the work described herein, it was unknown if such lyophilized formulations could even be made because successful lyophilization of biological molecules, and particularly live bacteria, is a challenging endeavour for several reasons. First, components used in the culture of bacteria can destabilize bacterial molecules even when freeze-dried. Second, bacterial viability can be impaired during the lyophilization process by interactions at the air/liquid interface and the solution/ice interface. Third, aggregation/clumping of the bacteria often occurs, leading to loss of function or viability and an inhomogenous and therefore unusable drug product. Fourth, crystal (ice) formation increases the salinity of the suspension tht can kill the bacteria present. And, fifth, dehydration can destabilize protein structure and activity.

There are also additional challenges involved in the large scale lyophilization of Bordetella-based (e.g., BPZE1-based) vaccines. For example, Bordetella species produce a large number of virulence factors that enable binding to epithelial cells, but these factors also cause the bacteria to adhere to one another which exacerbates the loss of function/viability caused by clumping and biofilm formation when grown to high cell densities in a bioreactor. Clumping or biofilm formation can lead to an inhomogenous product which, in turn, leads to significant loss of product on the filter during the tangential flow filtration (TFF) step. While clumping can be avoided by increasing agitation in the bioreactor, the increased shear forces associated therewith can lead to loss of viability. There is also an increasing loss of viability as the time between the harvest step and the start of lyophilization is increased. In the case of large scale manufacture where harvesting, concentrating, formulating, and then filling the product into lyophilization vials may take more than 20 hours, a significant loss of viability generally occurs. In addition, Gram negative bacteria such as Bordetella are particularly susceptible to loss of viability during the freezing step of the lyophilization process. BPZE1, in particular, has a thinner cell wall than its parent wild-type strain, and has mutations (a mutated pertussis toxin gene (ptx), a deleted dermonecrotic gene (dnt), and a heterologous ampG gene which replaces the native Bordetella ampG gene which might affect the ability of the bacteria to withstand lyophilization. See U.S. Pat. No. 9,180,178.

Accordingly, described herein are methods of making a lyophilized vaccine including live attentuated Bordetella bacteria as an active agent. These methods can include the steps of : harvesting Bordetella bacteria from a culture at an OD₆₀₀ between 0.4 and 1.6; mixing the harvested Bordetella bacteria with a lyophilization buffer comprising 5-65% by weight a cryoprotectant sugar and having a temperature between 2-35° C., wherein the ratio of harvested Bordetella bacteria to lyophilization buffer is between 5:1 and 1:5 by volume; lyophilizing the mixture of the Bordetella bacteria and the lyophilization buffer; wherein the hold time between the harvesting and lyophilization steps is less than 48 hours (e.g., less than 36 hours); and collecting the lyophilized Bordetella bacteria. The Bordetella bacteria can be a strain of Bordetella pertussis such as a BPZE strain. (e.g., BPZE1). In some variations of the methods, the Bordetella bacteria from cultures at an OD₆₀₀ between 0.4 and 1.0, or less than 1.0. The cryoprotectant sugar can be sucrose, and the lyophilization buffer can include a nutrient substrate such as glutamate.

The lyophilization step can include a pre-crystallization hold step wherein the mixture of the Bordetella bacteria and the lyophilization buffer is held at 0.1 to 10° C. above the crystallization temperature of the mixture for 0.5-10 hours prior to further cooling. The methods can also feature a step of concentrating the harvested Bordetella bacteria to an OD₆₀₀ of 1.0 -30.0 prior to the mixing step.

Also described herein are lyophilized vaccine products including live attentuated Bordetella bacteria made according to the methods described above and elsewhere herein. The lyophilized vaccine products can have a shelf life of at least two years when stored at 22.5° C. or lower, and at least 20% of the bacteria in the product remains viable after the lyophilization step. The collected lyophilized bacteria in the vaccine products can also feature the ability to prevent or reduce infection of a subject’s (e.g., a mammalian subject such as a human or mouse) respiratory tract with a pathogenic strain of Bordetella pertussis.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patents, and patent applications mentioned herein are incorporated by reference in their entirety. In the case of conflict, the present specification, including definitions will control. In addition, the particular embodiments discussed below are illustrative only and not intended to be limiting.

### DESCRIPTION OF THE DRAWINGS

FIG. 1 is a series of photographs of gels showing PCR analyses of loci of a lyophilized Bordetella bacteria (the BPZE1 strain of B. pertussis) formulation compared to a liquid formulation of BPZE1. E. coli ampG (FIG. 1A), B. pertussis ampG (FIG. 1B) and the B. pertussisdnt flanking regions (FIG. 1C) of two lots of the liquid BPZE1 formulation (lanes 1 and 2) and two lots of the lyophilized BPZE1 formulation (lanes 3 and 4), as well as a BPSM wild-type control (lane 5).

FIG. 2 is a graph showing the results of quantitative-PCR (q-PCR) amplification of the pertussis toxin (PTX) S1 subunit-coding DNA. Results for the S1 subunit gene of the liquid BPZE1 formulation (BPZE1 liquid), the lyophilized BPZE1 formulation (BPZE1 lyo), BPSM and BPSM-spiked lyophilized BPZE1 (Spiked) are shown.

FIG. 3 is a graph showing the microbiological stability (measured in CFUs) of the liquid BPZE1 formulation at various time points over 2 years storage at -70° C. The results for the liquid BPZE1 formulation at 10⁷ CFU/dose (middle line, low dose), 10⁸ CFU/dose (top line, middle dose) and 10⁹ CFU/dose (bottom line, high dose) are shown.

FIG. 4 is a graph showing the microbiological stability (measured in CFUs) of the lyophilized BPZE1 formulation over time. The lyophilized BPZE1 formulation at 10⁹ CFU/dose was stored at -20° C. ± 10° C. (top line), 5° C. ± 3° C. (middle line) and 22.5° C. ± 2.5° C. (bottom line) for two years, and CFUs were quantified at the indicated time points. The dotted and full lines represent the upper and lower limits of the specification indicated in Table 1 below.

FIG. 5 is a series of graphs showing the in vivo colonization kinetics of the lyophilized BPZE1 formulation compared to the liquid formulation in BALB/c mice which were intranasally administered 10⁵ CFU of the liquid BPZE1 formulation (black bars) or the reconstituted lyophilized BPZE1 formulation (gray bars) and sacrificed 3 h (day 0), 1 or 3 days thereafter. FIG. 5A shows a comparison of the CFU counts of the liquid BPZE1 formulation with those of the reconstituted lyophilized BPZE1 formulation reconstituted and administered immediately after lyophilization. FIG. 5B shows a comparison of the CFU counts of the liquid BPZE1 formulation with those of the reconstituted lyophilized BPZE1 formulation reconstituted 6 months after storage at -20° C. ± 10° C. (light gray bars), 5° C. ± 3° C. (medium gray bars) or 22.5° C. ± 2.5° C. (dark gray bars). FIG. 5C shows a comparison of the CFU counts of the liquid BPZE1 formulation with those of the reconstituted lyophilized BPZE1 formulation reconstituted 24 months after storage at -20° C. ± 10° C. (light gray bars), 5° C. ± 3° C. (medium gray bars) or 22.5° C. ± 2.5° C. (dark gray bars). The results are expressed as means +/- SEM. *, p < 0.05; ^{∗∗},p< 0.01; ***,p < 0.005; ns, not significant.

FIG. 6 is a series of graphs showing the potency of the lyophilized BPZE1 formulation compared to the liquid formulation in BALB/c mice were intranasally administered 10⁵ CFU of the liquid BPZE1 formulation (black bars) or the reconstituted lyophilized BPZE1 formulation (gray bars), or PBS as a mock control (white bars), and then challenged intranasally four weeks later with 10⁶ CFU of a virulent strain of B. pertussis (BPSM). CFUs present in the lungs were quantified 3 h (D0) and 7 days (D7) post challenge. FIG. 6A shows a comparison of the potency of the liquid BPZE1 formulation with that of the reconstituted lyophilized BPZE1 formulation reconstituted and administered immediately after lyophilization. FIG. 6B shows a comparison of potency of the liquid BPZE1 formulation with that of the reconstituted lyophilized BPZE1 formulation reconstituted 6 months after storage at -20° C. ± 10° C. (light gray bars), 5° C. ± 3° C. (medium gray bars) or 22.5° C. ± 2.5° C. (dark gray bars). FIG. 6C shows a comparison of the potency of the liquid BPZE1 formulation with that of the reconstituted lyophilized BPZE1 formulation reconstituted 24 months after storage at -20° C. ± 10° C. (light gray bars), 5° C. ± 3° C. (medium gray bars) or 22.5° C. ± 2.5° C. (dark gray bars). The results are expressed as means +/-SEM. ^{∗},p<0.005.

FIG. 7 is a graph showing a comparison of CFU counts of three different GMP runs after lyophilization using different methods as described in the Examples section below.

### DETAILED DESCRIPTION

Described herein are lyophilized formulations containing live attenuated Bordetella bacteria as the active agent which are stable for at least two years when stored at temperatures between -20° and 22.5° C., and which exhibit sufficient homogeneity (no bacterial clumping), bacterial viability and potency to be used as a live vaccine. Methods of making these lyophilized formulations are also described. The below described embodiments illustrate representative examples of these formulations and methods. Nonetheless, from the description of these embodiments, other aspects of the invention can be made and/or practiced based on the description provided below.

### General Methods of Making Lyophilized Formulations Containing Live Attenuated Bordetella Bacteria Suitable For Use As Vaccines

Lyophilized formulations containing live attenuated Bordetella bacteria are made by harvesting Bordetella bacteria from cultures at an appropriate growth phase, optionally concentrating the harvested Bordetella bacteria from the cultures, mixing the concentrated Bordetella bacteria with a lyophilization buffer containing a cryoprotectant sugar; and then lyophilizing the mixture of the Bordetella bacteria and the lyophilization buffer.

### Bordetella Bacteria

The Bordetella bacteria used in the compositions and methods described herein may be any suitable species or strain of Bordetellae. Bordetella species include Bordetella pertussis, Bordetella parapertussis, and Bordetella bronchiseptica. Preferred Bordetella bacteria are those that have shown activity as vaccines against infections disease (e.g., pertussis) or have other beneficial prophylactic or therapeutic effects (e.g., reduction of inflammation or treatment of allergy). A number of live, attenuated B. pertussis strains have been made which are effective in preventing or reducing the pathology associated with pertussis, other infectious diseases, or have other beneficial prophylactic or therapeutic effects are preferred for use in the methods and compositions described herein. These include the various BPZE strains such as BPZE1 (described in U.S. Pat. No. 9,180,178; and deposited with the Collection Nationale de Cultures de Microorganismes in Paris, France on Mar. 9, 2006 under accession number CNCM I-3585), and variants thereof such as BPZE1 modified to express a hybrid protein including the N-terminal fragment of filamentous haemagglutinin (FHA) and a heterologous epitope or antigenic protein or protein fragment (described in U.S. Pat. No. 9,528,086), adenylate cyclase-deficient BPZE strains such as BPAL10 (described in U.S. Pat. No. 10/369,207; and deposited with the National Measurement Institute, 1/153 Bertie Street, Port Melbourne, Victoria, Australia 3207 on Oct. 23, 2015 under accession number V15/032164) and BPZE1AS (described in WO2020049133; and deposited with Collection Nationale de Cultures de Microorganismes on Sep. 4, 2018 under accession number CNCM 1-5348), pertactin-deficient BPZE strains such as BPZE1-P (described in U.S. Pat. No. 11,065,276; and deposited with Collection Nationale de Cultures de Microorganisms on Dec. 12, 2016 under accession number CNCM-I-5150), and Fim2- and Fim3-producing BPZE strains such as BPZElf3 (described in U.S. Pat. Application No. 16/848,793; and deposited with Collection Nationale de Cultures de Microorganisms on Oct. 11, 2017 under accession number CNCM I-5247).

### Pre-Lyophilization Processing of Bordetella Bacteria

The methods of making lyophilized vaccine products including live attenuated Bordetella bacteria begin with culturing and then harvesting the Bordetella bacteria from a bioreactor. Suitable media and culture conditions are described in the Examples section below. Harvesting the cultured bacteria is performed by standard methods. Because initial studies unexpectedly showed that Bordetella bacteria like BPZE1 are especially prone to aggregation/clumping in culture, to avoid the loss of viability due to this aggregation/clumping it is preferred that harvesting be performed when the culture reaches an OD₆₀₀ between 0.4 and 1.6; 0.5-1.5, 0.6-1.4, 0.7-1.3, 0.8-1.2, 0.9-1.1, 1.0, or less than 1.0 (e.g., at 0.2, 0.3, 0.4, 0.5, 0.6, 0.7, 0.8, or 0.9). After the Bordetella bacteria have been harvested, they may be optionally concentrated (e.g., to meet final CFU/dose requirements) and/or subjected to diafiltration to reduce salt or exchange buffer. For example, the harvested Bordetella bacteria can be concentrated to an OD₆₀₀ of 1.0 - 30.0 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 +/- 0, 0.1, 0.2, 0.3, 0.4, or 0.5) prior to the mixing step. After harvesting and concentrating/diafiltration (if performed), the bacteria are then mixed with a suitable lyophilization buffer. When mixed with the bacteria the lyophilization buffer is generally at a temperature between 2-35° C. (e.g., between 4-30° C., between 8-25° C., between 10-20° C., or 4+/-1, 2, or 3° C.). A suitable cryoprotectant is included in the lyophilization buffer (or added in the mixing step) at a weight ratio of 5-65% (e.g., 10, 15, 20, 25, 30, 35, 40, 45, 50, 55, or 60 +/- 0, 1, 2, 3, 4, or 5%) of the lyophilization buffer. Based on a comparison of different cryoprotectants, cryoprotectant sugars (particularly sucrose) are preferred. The ratio of Bordetella bacteria to lyophilization buffer in the mixture is between 5:1 and 1:5 (e.g., 5:1, 4:1, 3:1, 2:1, 1:1, 1:2, 1:3, 1:4, 1:5, between 4:1 and 1:4, between 3:1 and 1:3, or between 2:1 and 1:2) by volume. The time between harvesting and the start of lyophilization should be less than 48 hours (e.g., less than 44, 40, 36, 32, 28, 24, 20, or 16 hours) to avoid significant losses in viability.

### Lyophilization

The prepared mixture of bacteria and lyophilization buffer is then aliquoted into lyophilization containers (e.g., glass vials) containing between 5 X 10⁶ to 1 X 10¹⁰ (e.g., 1 X 10⁶, 5 × 10⁶, 1 X 10⁷, 5 × 10⁷, 1 X 10⁸, 5 X 10⁸, 1 X 10⁹, 2 X 10⁹, or 3 X 10⁹ +/- 10, 20, 30, 40, or 50%) CFU of bacteria. The filled containers are then placed in a lyophilizer and the lyophilization process is started. Primary drying can be performed in the range of -40-0° C. (e.g., at -34° C.) under suitable pressure (e.g., between 50-250 microbar or 100 +/- 0, 10, 20, 30, 40, 50, 60, 70, 80, or 90 microbar). This primary drying step is typically continued until the pirani and the capacitance manometer readings converged, indicating that sublimation had ended. Primary drying can be followed by a ramp of temperature, e.g., from the primary drying temperature to a secondary drying temperature of between +10 to +40° C. (e.g., +30 =/- 0, 1, 2, 3, 4, 5, 6, 7, 8, 9, 10° C.) over several hours (e.g., 6, 7, 8, 9, 10, 11, 12, 13, 14, 15, 16, 17, or 18 hours), followed by holding the temperature at the secondary drying temperature until the pressure rise increased less than 10 microbar after closing the valve to the condensor chamber (indicating that the product was dry). The containers are then stoppered at 100 to 1000 mBar using dry compressed air or 100% nitrogen or any mixture of both, cooled (e.g., to +4° C.), unloaded, and then capped (e.g., with an aluminum cap).

For large scale production, the lyophilization step preferably includes a pre-crystallization hold step to reduce vial-to-vial viability. Ice crystal formation means that the dissolved components of the lyophilization buffer increase in molarity, including the salts. The high salt concentration is likely to damage the outer membrane of B. pertussis, or any other bacterium, yeast, fungus or virus, so that duration of the phase in which high salt concentrations are present should be shortened if possible. Because glass vials conduct heat/cold very poorly and typically contact the lyophilizer shelf at only 3 points, during freezing, this poor conduction leads to inhomogenous cooling of vials such that the liquid in some vials will have initiated crystal formation while the liquid in others will remain liquid longer. As described below in the Examples section, when a lyophilization buffer is cooled very slowly, ice crystal formation in the vials took place abruptly at a specific temperature above the glass transition temperature (Tg′). If held at this specific temperature (the crystallization point), most vials showed abrupt crystal formation within less than a minute of each other. On the other hand, when then cooling step was not subject to a hold period, ice crystal formation among the different vials can vary by more than an hour - leading to large differences in exposure to high salt concentrations and therefore large differences in viability among the vials.

The introduction of a pre-crystallization hold step prior to freezing to the Tg′ is as follows. Of a given lyophilization buffer the crystallization temperature is determined by slowly cooling the buffer and noting the temperature at which the onset of crystallization occurs. The pre-crystallization hold step can be defined as a hold step of half an hour to several (e.g., 0.5, 0. 6, 0.7, 0.8, 0.9, 1, 2, 3, 4, 5, 6, 7, or 8) hours, depending on the size of the lyophilizer, the size of the vials and the amount of liquid in the vials, at 0.1 to several (e.g., 0.1, 0.2, 0.3, 0.4, 0.5, 0.6, 0.7, 0.8, 0.9, 1, 2, 3, 4, 5, 6, 7, or 8) °C above the crystallization point, depending on the variability of the temperature of the cooling liquid running through the lyophilizer shelves.

### EXAMPLES

### Example 1- Materials and Methods

### Bacterial Strains and Growth Conditions

Virulent B. pertussis BPSM (Menozzi et al., Infect Immun 1994, 62:769-778) was grown at 37° C. on Bordet-Gengou (BG) agar containing 100 µg/ml streptomycin and supplemented with 1% glycerol and 10% defibrinated sheep blood as described (Mielcarek et al., PLoS Pathog 2006; 2: e65). After growth, the bacteria were harvested by scraping the plates and resuspending them in phosphate-buffered saline (PBS) at the desired density. The BPZE1 vaccine strain (Mielcarek et al., PLoS Pathog 2006; 2: e65) Working Cell Bank (WCB) was grown in fully synthetic Thijs medium (Thalen et al., Biologicals 2006, 34:213-220) under agitation. After addition of 20% vol/vol of 86% glycerol and filling in 1.5 mL aliquots in cryo-vials, the WCB was stored at -70° C. until further use, as described (Thorstensson et al., PLoS ONE 2014; 9, e83449; and Jahnmatz et al., Lancet Infect Dis 2020, 20:1290-1301).

### Fermentation of BPZE1

The WCB with a volume of 1.5 ml was inoculated in Erlenmeyer flasks containing 28.5 ml of Thijs medium (Thalen et al., Biologicals 2006, 34:213-220). The second pre-culture, consisting of a 2-L Erlenmeyer with 0.5 L Thijs medium, was inoculated at an OD₆₀₀ of 0.1, which was in turn used as inoculum for 5 x 2-L flasks with 0.5 L Thijs medium each. The 5 cultures were pooled and added to a 50-L bioreactor (Sartorius, 50 L SUB) with 20 L Thijs medium so that the bioreactor started at an OD₆₀₀ of 0.1. The fermentation was performed at 35° C., dissolved oxygen was controlled at 20% using compressed air supplied through the sparger, and the pH was controlled at pH 7.5 using 0.2 M lactic acid. All product contact materials, such as the culture and medium flasks, containers, tubing, filters, connectors, as well as the bioreactor were single use. After reaching the target OD₆₀₀ of 1.1 - 1.4, a sample of 8 L culture was concentrated and/or diafiltered to an OD₆₀₀ as specified using hollow fiber tangential flow filtration (TFF; 750 kDA mPES membrane 1400 cm², Spectrum) at a maximum transmembrane pressure of 0.3 bar.

### Lyophilization of BPZE1

Initial culture and lyophilization development resulted in a lyophilization buffer and lyophilization cycle at small scale. For all larger scale cultures the lyophilization buffer, cooled to +4° C., was added in a 1:1 ratio to the bacterial suspension, using 100 g/L sucrose as main cryoprotectant. The resulting formulated mixture was then filled in DIN 2R vials with a 13 mm bromobutyl lyophilization stopper and lyophilized using a conservative cycle, consisting of primary drying at -34° C. at 100 microbar until the pirani and the capacitance manometer readings converged, indicating that sublimation had ended. Primary drying was followed by a ramp from -34 to +30° C. in 12 hours, followed by holding the temperature at 30° C. until the pressure rise increased less than 10 microbar after closing the valve to the condensor chamber, indicating that the product was dry. After stoppering the vials were cooled to +4° C. until unloading, followed by capping the vials with an aluminum cap.

### Plate Count

The enumeration of the Colony Forming Units (CFU) was performed by plating 1, 2 and 5 times 10-fold dilutions of the formulation samples on Bordet Gengou agar plates supplemented with 15% sheep blood. All dilutions were plated in triplicate so that on average 9 plates were counted to obtain a single CFU result. The specification of the formulation after lyophilization was set to 0.2 - 4.0 × 10⁹ CFU/ml.

### Microbial Safety Tests on Drug Substance and Formulations

The absence of Staphylococcus aureus, Pseudomonas aeruginosa and bile tolerant organisms was tested according to the United States Pharmacopoeia, test 62 (USP<62>) (United States Pharmacopoeia, USP42-NF37, 2019), while the purity of both the drug substance and the formulation was tested according to USP<61>. All runs complied with both USP safety tests.

### Mouse Colonization and Potency Assays

BALB/c mice were purchased from Charles Rivers and kept at an animal facility under specific pathogen-free conditions. For colonization assays, the various BPZE1 suspensions were diluted to 10⁵ CFU per 20 µl, which were nasally administered to six-week old BALB/c mice. The mice were sacrificed 3 h, 24 h or 3 days after infection, and nasal homogenates were prepared as described (Solans et al., Mucosal Immunol 2018, 11:1753-1762) and then plated in ten-fold serial dilutions onto BG blood agar pates and incubated at 37° C. for 3-5 days to quantify colonization by CFU counting. To determine the potency of the various BPZE1 formulations, six-week old wild BALB/c mice were intranasally vaccinated with 10⁵ CFU of BPZE1 or received PBS intranasally as described (Debrie et al., Vaccine 2018, 36:1345-1352). Four weeks later, the mice were challenged intranasally with 10⁶ CFU of virulent BPSM. Lung colonization was determined 3 h and 7 days post challenge.

### Genetic Stability Assays

The genetic stability of the various BPZE1 preparations was evaluated by polymerase chain reaction (PCR) targeting the dnt and ampG genes as described (Feunou et al., Vaccine 2008, 26:5722-5727). The pertussis toxin (PT) S1 subunit gene ptxA was analyzed by quantitative PCR (Q-PCR) for the absence of reversion of the two codon changes introduced to inactivate PT (Mielcarek et al., PLoS Pathog 2006; 2:e65). Approximately 10¹⁰ CFU of the BPZE1 preparations were harvested by centrifugation and suspended in buffer B1 (Qiagen, #19060), containing RNaseA and proteinase K, and incubated at 37° C. for 30 min. The bacteria were then lysed in lysis buffer for 30 min at 50° C. and applied to a Qiagen genomic-tip 100/G column.

After washing and elution as recommended by the manufacturer, the DNA was precipitated with isopropanol (CarloErba), centrifuged at 5,000 × g for 15 min, washed with ice cold 70% ethanol, air dried for 10 min and resuspended in 100 µl bi-distilled water. The DNA concentration was measured using a NanoDrop 2000c spectrophotometer. One µl BPZE1, BPSM or BPSM-spiked BPZE1 DNA corresponding to 10⁷ genome copies was mixed with 19 µl of LightCycler 480 SYBR Green I Master mix containing 0.5 µM of primer pairs in 96-well LightCycler 480 plates. The plates were sealed with specific plastic film, transferred to the LightCycler 480 and subjected to 15 min incubation at 95° C., followed by 1 to 40 cylces of denaturation for 15 seconds at 95° C., annealing for 8 seconds at 68° C. and 18 seconds of elongation at 72° C. The data were then analysed using the LightCycler 480 software release 1.5.0. To control for sensitivity of the assay able to detect one potential reversion among 10⁶ copies of the genome, 10 copies of BPSM DNA were mixed with 10⁷ copies of BPZE1 DNA. All primers were purchased from Eurogentec (Liège, Belgium).

### Example 2- Results

### BPZE1 Formulation Development

B. pertussis produces a number of virulence factors that enable binding to epithelial cells as well as to each other, and is capable of biofilm formation. In a bioreactor, biofilm formation leads to bacterial clumping and therefore to an inherently inhomogenous vaccine formulation. Clumping in the bioreactor can be avoided by increasing agitation, but too high shear forces during fermentation or ultrafiltration lead to cell damage, which translates into low survival after lyophilization. In the 20-L bioreactors with 8 L medium, run at 400 RPM using a 6 blade Rushton impeller, post-lyophilization survival did not exceed 45%, while in the 50-L bioreactor with 20 L medium, run at 150 RPM using a 3-blade marine impeller showed post-lyophilization survival of up to 65% under similar conditions (data not shown).

At 8 L bioreactor scale, suspension OD_{600S} of 0.5 showed little clumping, but poor survival after lyophilization compared to OD_{600S} of >1.0. Therefore, all the subsequent cultures were harvested at an OD₆₀₀ of 1.1 - 1.6. These OD_{600S} correspond to approximately 50 to 80% of the maximum OD₆₀₀, well before all medium substrates were consumed, so that the bacteria were in a physiological state that results in high survival after lyophilization. In order to halt cellular metabolism during the period between the harvest and freezing on the shelf of the lyophilizer, the addition of cold lyophilization buffer was found to be suitable.

To minimize the impact of bioreactor and TFF geometry on post-lyophilization survival, all 50-L bioreactors were run using the same conservative conditions during fermentation and ultrafiltration, compromising between minimizing shear stress while avoiding clumping.

### Lyophilization Buffer Development

The manufacturing process development for the formulation consisted of developing a lyophilized formulation, including a lyophilization buffer and a matching lyophilization cycle, as well as verifying that the developed process does not interfere with the biological activity of the BPZE1 formulation. It is especially important that the lyophilized formulation maintains its ability to colonize the nose (adherence assay) and to reduce the bacterial burden in the lungs by at least two orders of magnitude in the murine protection assay (potency assay). The target formulation attributes are shown in Table 1.

TABLE 1

Target formulation attributes for the lyophilized BPZE1 formulation.

| | | |
|---|---|---|
| Attribute | Target Specification | Method |
| Hold time prior to lyophilization | 24 - 48 hour | Plate count on Bordet Gengou plates |
| Appearance upon reconstitution | opaque liquid, no visible clumping | visual inspection USP<790> |
| Shelf life post lyophilization | 0.2 - 4x10⁹ CFU/ml for ≥2 year | Plate count on Bordet Gengou plates |
| Survival post lyophilization | ≥20% | Plate count on Bordet Gengou plates |
| Residual moisture content (RMC) | ≤2.5% | Karl Fischer, USP<921> (United States Pharmacopoeia, USP42-NF37, 2019) |
| Glass transition temperature (Tg) | ≥35° C. | Differential scanning calorimetry |
| Adherence & colonization of murine nasal cavity | comparable to liquid phase Ib formulation after 3 days colonization | Homogenization of murine nasal cavity followed by plating the homogenate on Bordet Gengou plates |
| Potency assay | Reduction in bacterial burden of ≥100-fold compared to controls | Homogenization of murine lungs 3 hours and 7 days after BPSM challenge followed by plating the homogenate on Bordet Gengou plates |

The formulation of the lyophilization buffer was based on commonly used cryoprotectants, containing 5 to 10% sucrose or trehalose, sometimes in combination with other cryoprotectants such as hydroxy ethyl starch (HES) or sodium glutamate (MSG). A single bacterial suspension was used to generate all formulations shown in Table 2. All formulations showed a residual moisture content (RMC) below the 2.5% target and a glass transition temperature (Tg) above the 35° C. target. Sucrose appeared superior over trehalose as cryoprotectant when used alone. The addition of HES or MSG to trehalose or sucrose did not enhance survival. Repeat experiments with sucrose and trehalose showed similar results, although the absolute survival percentages varied between experiments. Therefore, 10% sucrose was chosen for further development.

TABLE 2

Residual moisture content, glass transition temperature and bacterial survival as function of lyophilization buffer conditions.

| Threhalose | Sucrose | HES¹ | Na-glutamate | RMC (%) | Tg (°C) | survival (%)² |
|---|---|---|---|---|---|---|
| - | 5% | - | - | 1.0 | 43 | 44 |
| - | 10% | - | - | 1.8 | 36 | 38 |
| - | 5% | 5% | - | 0.2 | 48 | 25 |
| - | 10% | 7% | - | 0.3 | 46 | 44 |
| 5% | - | - | - | 0.6 | 65 | 24 |
| 10% | - | - | - | 0.4 | 53 | 33 |
| 5% | - | 5% | - | 0.2 | 54 | 24 |
| 10% | - | 7% | - | 0.3 | 54 | 26 |
| 5% | - | 7% | 1% | 0.3 | 54 | 22 |
| 10% | - | 7% | 2% | 0.3 | 57 | 31 |

¹HES, Hydroxy ethyl Starch; RMC, residual moisture content; Tg, glass transition temperature.
²Survival is expressed as percentage of CFU comparing the pre- and post-lyophilization content of the vials.

An overview of the various runs, carried out all in the same type bioreactor, is shown in Table 3, indicating the manufacturing method, such as direct dilution of the culture in lyophilization buffer, concentration and diafiltration of the culture, followed by dilution with lyophilization buffer and concentration of the culture followed by dilution with lyophilization buffer. Various diafiltration buffers were tried to wash the concentrated bacterial suspension, including Thijs medium without NaCl and Tris, and without Thijs supplement, with variable success. The main reason to diafilter the BPZE1 drug substance was to reduce the salt content coming from the medium: 1.66 g/L NaCl and 0.765 g/L Tris, since the presence of these salts resulted in a slower lyophilization cycle than without the salts. However, in all drug substances that were concentrated and diafiltered some degree of clumping was observed (Table 3).

TABLE 3

Overview of the various runs carried out in a 50-L single use bioreactor with 20 L medium, comparing different harvest methods.

| Batch: | | Run 1¹ | | Run 2² | Run 3³ | Run 4³ | Run 5³ | Run 6³ | | Run 7³ |
|---|---|---|---|---|---|---|---|---|---|---|
| | | 1a | 1b | | | | | 6a | 6b | |
| Manufactured by: | | direct dilutio n4 | concentration & diafiltration⁵ | | | | | | direct dilutio n4 | concen -tration 6 |
| Test | Proposed Specificat ion | Result | Result | Result | Result | Result | Result | Result | Result | Result |
| Hold time | 24-48 hours | 6 | 6 | 16 | 28 | 31 | 26 | 28 | 28 | 32 |
| Homogen eity | Homogen ous suspension | not tested | not tested | minor clumpi ng | minor clumpi ng | minor clumpi ng | severe clumpi ng | severe clumpi ng | pass | pass |
| Plate count pre-lyophiliza tion | 0.4-8.0 x10⁹ CFU/ml | 1.1 ×10⁹ | 2.4 ×10⁹ | 6.8 ×10⁹ | 3.2×10 9 | 1.8×10⁹ | 7.7 x10⁹ | 2.4 x10⁹ | 2.2 ×10⁹ | 8.3 ×10⁹ |
| Plate count post lyophiliza tion | 0.2 - 4.0 ×10⁹ CFU/ml | 0.7 ×10⁹ | 1.1 ×10⁹ | 3.2 ×10⁹ | 0.6 ×10⁹ | 0.3 ×10⁹ | 0.4 ×10⁹ | 0.2 ×10⁹ | 0.4 ×10⁹ | 1.9 ×10⁹ |
| Survival % | - | 64 | 46 | 47 | 19 | 17 | 5 | 8 | 18 | 23 |

¹) Run 1 was filled <50 vials, lyophilization was started with <6 h hold time.
²) Run 2 was filled <700 vials, lyophilization was started with <16 h hold time.
³) Runs 3 to 7 were harvested, lyophilized in 2000 to 7000 vials per formulation and lyophilization was initiated after between 24 and 36 h hold time.
⁴) Direct dilution: dilution of the culture 1:1 with lyophilization buffer
⁵) Concentration & diafiltration: concentration of the culture followed by diafiltration and 1:1 dilution with lyophilization buffer,
⁶) Concentration: concentration of the culture followed by 1:1 dilution with lyophilization buffer.

Thijs medium is chemically defined and consists of components that are all generally regarded as safe. Therefore, there is no need to remove these components from the BPZE1 formulation from a quality perspective. Cultures that were either directly diluted with lyophilization buffer (Table 3, Runs 1a and 6b) or were concentrated and subsequently diluted with lyophilization buffer (Table 3, Run 7) did not show any signs of clumping directly after the harvest and just before filling. To meet the CFU target of the formulation, the culture was concentrated to an OD₆₀₀ of 5.0, followed by diluting the bacterial suspension 1:1 with cold lyophilization buffer (Table 3, Run 7).

The hold time between harvest and the start of lyophilization had a major impact on bacterial survival both before and after lyophilization. The first runs showed high post lyophilization survival of 64% using 1:1 direct dilution of the culture with lyophilization buffer and (Table 3, Run 1a), while the diafiltered cultures showed 46% and 47% survival (Table 3, Run 1b and Run 2). These formulations were lyophilized within 16 hours after harvest and formulation, while all subsequent runs were lyophilized between 26 and 32 hours after harvest. Runs 6b and 7 were tested for viability of the bacteria in the formulation directly after formulation and after 48 hours of storage at +4° C. Both formulations had lost approximately half the CFU, which explains the relatively poor survival of 18 and 23% for runs 6b and 7, respectively (Table 3). Thus, the storage duration prior to lyophilization had a significant impact on post-lyophilization survival, since otherwise the survival between Run 1a and Run 7 would have been more similar.

### Genetic Comparison of Liquid and Lyophilized BPZE1 Formulations

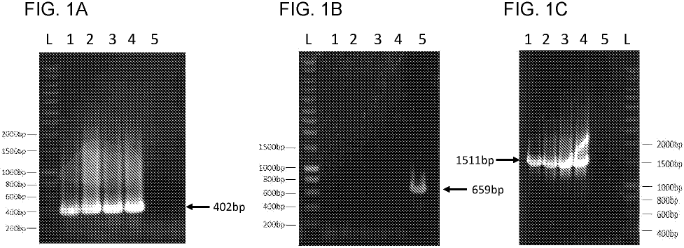

The lyophilized formulation was compared to the liquid formulation stored at -70° C. to verify that the mutations introduced into the B. pertussis genome to generate BPZE1 were conserved, in particular the deletion of the dnt gene, the replacement of the B. pertussis ampG gene by the E. coli ampG gene and the presence of the two mutated codons in the PT S1 subunit gene. The first two genetic modifications were verified by PCR as described in Feunou et al., Vaccine 2008, 26:5722-5727. The presence of the E. coli ampG gene was detected by the amplification of a 402-bp fragment corresponding to an internal fragment of the E. coli ampG gene. The two lyophilized BPZE1 formulations and the two liquid BPZE1 formulation controls yielded the expected 402-bp fragment, whereas this was not seen in the BPSM control sample (FIG. 1A). Conversely, a 659-bp fragment corresponding to the B. pertussis ampG gene was amplified in the BPSM control sample, but not in any of the BPZE1 formulations (FIG. 1B), indicating that both the liquid and the lyophilized BPZE1 formulations lacked B. pertussis ampG, but contained E. coli ampG. The deletion of the dnt gene was shown by the amplification of a 1,511-bp fragment resulting from a PCR using primers that flank the deleted dnt gene. The two lyophilized BPZE1 formulations and the two liquid BPZE1 formulation controls yielded the expected 1,511-bp fragment, whereas this was not seen in the BPSM control sample (FIG. 1C).

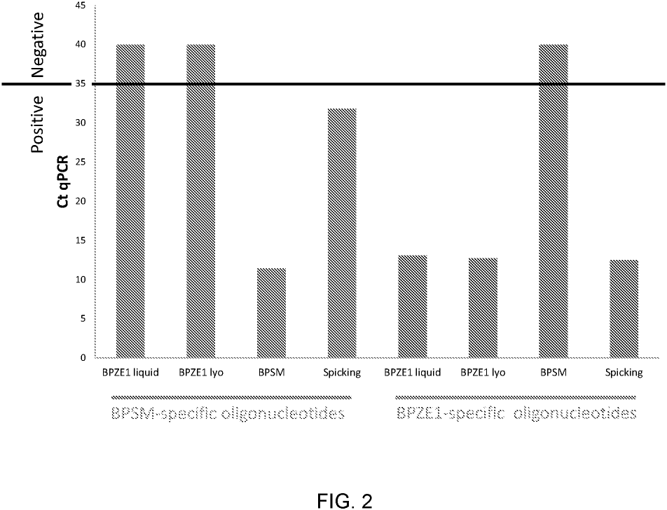

To verify the presence of the 2 mutated codons in the PT S1 gene, a quantitative PCR method was developed, which is able to detect 1 copy of the wild type gene among 10⁶ copies of the mutated gene. For this purpose, 10⁷ copies BPZE1 DNA, and 10⁷ copies BPZE1 DNA spiked with 10 copies of BPSM DNA were subj ected to qPCR using BPSM- or BPZE1-specific oligonucleotides. 10⁷ copies BPSM DNA served as control. The threshold of positivity was set at 35 qPCR cycles. The lyophilized BPZE1 formulation and the liquid BPZE1 formulation showed indistinguishable amplification patterns, i.e., no amplification was observed with the BPSM-specific primers, while amplicons were detected with Cp values between 12.21 and 13.32 when using BPZE1-specific primers. In contrast, BPSM DNA was amplified with the BPSM-specific primers, but not with the BPZE1-specific primers, while spiked BPZE1 DNA was amplified with both primer pairs (FIG. 2). These results indicate that BPZE1 had retained the codon modifications and that no reversion occurred at a frequency higher than ⅒⁶.

### Microbiological Stability

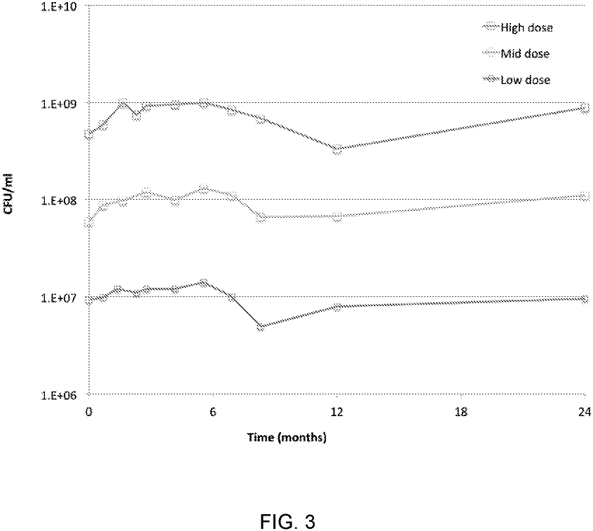

The stability of the liquid BPZE1 formulations stored at -70° C. was followed up for 2 years storage at -70° C. at three different formulations, 10⁷ (low dose), 10⁸ (middle dose) and 10⁹ CFU/dose (high dose). As shown in FIG. 3, the liquid BPZE1 formulation stored at -70° C. was stable for a minimum of 2 years at each dose tested.

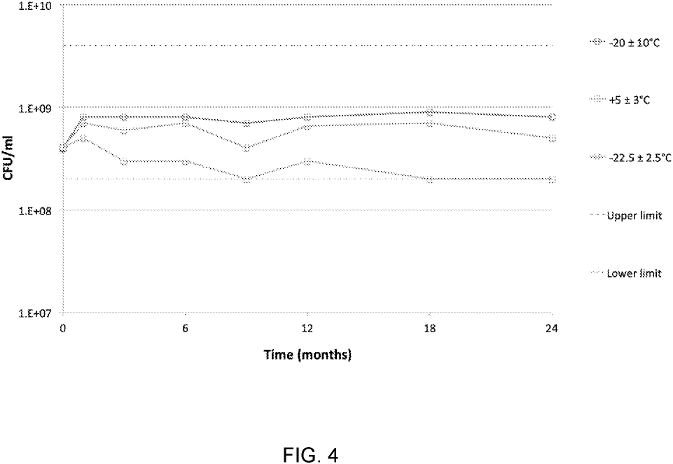

We tested the microbiological stability of the lyophilized BPZE1 formulation formulated at 10⁹ CFU/dose at -20° C. ± 10° C., 5° C. ± 3° C. and 22.5° C. ± 2.5° C. As shown in FIG. 4, at all tested temperatures, the 10⁹ CFU/dose lyophilized BPZE1 formulation met the CFU specification, even when stored at 22.5° C. ± 2.5° C. for at least 2 years. Whereas no sign of CFU loss was seen in the formulation stored at -20° C. ± 10° C. or 5° C. ± 3° C., the formulation stored at 22.5° C. ± 2.5° C. showed some loss in viability during the first months of storage, but remained stable thereafter up to at least 2 years. Nevertheless, even in this case the CFU counts remained within specification. The stability data of Run 7, which was produced by concentrating the culture and diluting it with lyophilization buffer, was similar to that of Run 6, albeit at a higher CFU count due to the concentration step prior to adding the lyophilization buffer.

### Biological Stability

The biological stability of the lyophilized BPZE1 formulation was evaluated in two different mouse assays: an in-vivo nasal adherence & colonization assay and a potency assay. In each of these assays the performance of the BPZE1 formulation stored at the various temperatures was compared with that of the original liquid formulation of BPZE1, stored at -70° C.

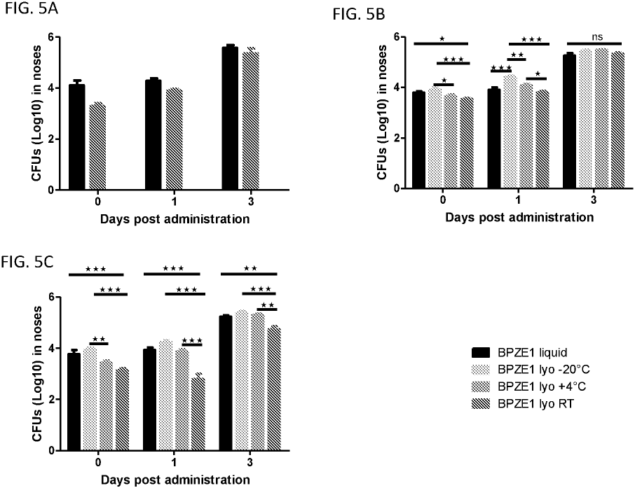

To quantify the kinetics of in-vivo nasal adherence and colonization, mice were intranasally inoculated with approximately 10⁵ CFU of reconstituted lyophilized BPZE1 formulation stored at different temperatures or the liquid BPZE1 formulation control. Three hours, one day and 3 days after administration, mice were sacrificed and CFU present in the nasal homogenates were counted. First, the effect of lyophilization and the composition of the lyophilization buffer was tested by comparing the liquid formulation with the lyophilized formulation immediately after lyophilization. As shown in FIG. 5A, both formulations adhered to and colonized the murine nasal cavity equally well, as there was no statistically significant difference between the liquid formulation and the lyophilized formulation. The lyophilized formulation was then stored for 2 years at -20° C. ± 10° C., 5° C. ± 3° C. or 22.5° C. ± 2.5° C., and adherence and colonization kinetics were evaluated after 6 months (FIG. 5B) and 24 months (FIG. 5C) of storage and compared to those of the liquid formulation. Although after storage of 6 months, the material stored at -20° C. ± 10° C. adhered slightly better at day 0 and colonized faster 1 day after administration than the material stored at the other temperatures, this difference was no longer detected 3 days after administration (FIG. 5B). However, after 24 months of storage, the lyophilized formulation stored at 5° C. ± 3° C. and 22.5° C. ± 2.5° C. adhered slightly less well at day 0 and colonized slightly slower at both 1 and 3 days after administration than the formulation stored at -20° C. ± 10° C. (FIG. 5C).

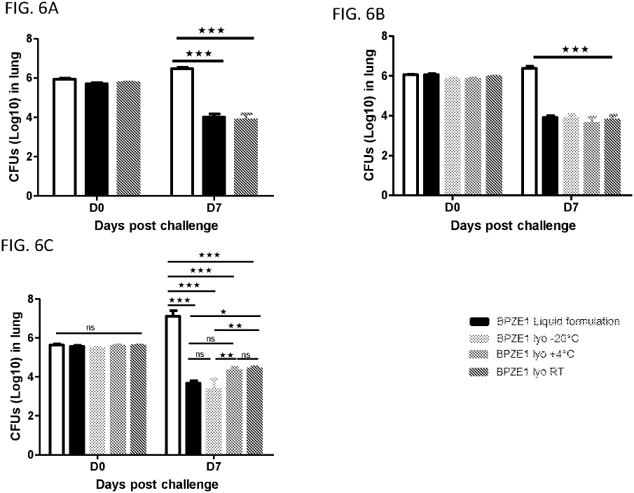

To evaluate the potency of the BPZE1 formulation after storage at different temperatures, mice were intranasally immunized with 10⁵ CFU of the reconstituted, lyophilized BPZE1 formulation or with the BPZE1 liquid formulation control, followed by an intranasal challenge with virulent BPSM. Mice were sacrificed 3 hours or 7 days after the BPSM challenge to evaluate the bacterial load in the lungs. First, the liquid formulation was compared to the lyophilized formulation tested immediately after lyophilization. Both formulations protected mice equally well, as the CFUs in the lungs decreased by two orders of magnitude between day 0 (3 h) and day 7 after challenge, whereas in the lungs of the non-vaccinated mice the bacterial load increased between day 0 and day 7 (FIG. 6A). Storage of the lyophilized formulation for 6 months at all temperatures tested did not affect the vaccine potency, as 7 days after challenge unvaccinated mice carried approximately ten-fold more BPSM bacteria in their lungs than 3 hours post-infection, whereas all vaccinated mice showed an approximately 100-fold reduction of CFU in their lungs, compared to those of the non-vaccinated controls (FIG. 6B). No statistical difference was seen between mice immunized with the liquid BPZE1 formulation and those immunized with the lyophilized BPZE1 formulation, and no influence of the storage temperature could be detected. Thus, despite the slightly lower adherence on day 0 and the slower colonization of the mouse nasal cavity on day 1 by lyophilized BPZE1 formulation stored at 5° C. ± 3° C. or 22.5° C. ± 2.5° C. compared to the product stored at -20° C. ± 10° C., this had no effect on the lyophilized formulation’s ability to provide protection against a BPSM challenge, when stored for 6 months.

After 24 months of storage, the lyophilized formulations stored at 5° C. ± 3° C. or at 22.5° C. ± 2.5° C. showed a slight, but significant decrease in potency, compared to the lyophilized formulation stored at -20° C. ± 10° C. (FIG. 6C). However, compared to the non-vaccinated mice, those that had received the formulation stored at 5° C. ± 3° C. or at 22.5° C. ± 2.5° C. still showed an almost 1000-fold decrease in bacterial burden in the lungs.

Together these data show that after storage of the lyophilized BPZE1 formulation between -20° C. ± 10° C. and 22.5° C. ± 2.5° C. for at least 2 years, lyophilized BPZE1 maintained its ability to colonize the nasal cavity and its ability to protect mice from virulent B. pertussis challenge within specification.

### Discussion

In previous studies a single nasal administration of BPZE1 was shown to provide protection against B. pertussis challenge in mice (Mielcarek et al., PLoS Pathog 2006; 2:e65; and Solans et al., Mucosal Immunol 2018, 11:1753-1762) and non-human primates (Locht, et al., J Infect Dis 2017, 216: 117-124), and was found to be safe, even in severely immunocompromised animals, such as IFN-y receptor KO mice and MyD88-deficient mice. BPZE1 was also shown to be safe and immunogenic in humans in phase I and phase II clinical trials.

All pre-clinical and clinical studies so far have been performed with a liquid formulation of BPZE1 that had to be stored at ≤-70° C., a temperature at which it was stable for at least 2 years at 10⁷ CFU/ml, 10⁸ CFU/ml and 10⁹ CFU/ml (FIG. 3). However, storage at -70° C. is incompatible with further clinical and commercial development. Herein it is described that a lyophilized BPZE1 formulation can be obtained, that is stable for at least 2 years at -20° C. ± 10° C., 5° C. ± 3° C. or 22.5° C. ± 2.5° C.

Several product target attributes were formulated prior to initiating BPZE1 process development, as listed in Table 1. A post-lyophilization survival of 20% was targeted, as this was the survival percentage of the liquid BPZE1 formulation used in the phase 1 trials [11, 12]. The target for the lyophilized BPZE1 formulation was that the CFU counts should remain between 0.2 and 4 × 10⁹ CFU/ml over at least a 2-year storage period at +4° C.

The survival after lyophilization of a live organism depends on the lyophilization cycle, lyophilization buffer and the physiological state of the organism prior to lyophilization. These parameters are likely interdependent. However, it became apparent that survival of BPZE1 after lyophilization also depends on the culture and harvest conditions, in particular shear stress and the harvest optical density had a significant impact on post-lyophilization survival as well as on the clumping behavious of the suspension to be harvested.

The critical importance of the hold time of the liquid bacterial suspension between harvest and start of lyophilization also became apparent during the actual production runs. While initial runs in which the start of lyophilization followed the harvest within 16 hours yielded 46 to 64% bacterial survival, hold times between 26 h and 32 h resulted in a reduction in survival to approximately 20%. Evaluating the survival after a hold time of 24 h to 48 h is particularly important for large-scale production, since harvesting, concentrating and formulating the bacterial suspension, and especially filling >200,000 vials per batch likely takes between 24 and 48 hours.

The RMC of the lyophilized formulation was consistently below 2.5% which is generally compatible with long term stability at 5° C. or lower. However, the relation between temperature and post-lyophilization survival is determined by the Tg, which is the temperature at which the remaining water in the lyophilized product becomes mobile again, leading to accelerated loss of viability. A target Tg was set at ≥35° C. for logistical and supply chain reasons, since relatively brief exposure (from hours to several days) of the formulation to ambient, yet controlled temperatures, does not significantly affect the formulation, as confirmed by the stability of the lyophilized formulation for 2 years at +22.5 ± 2.5° C.

The manufacturing process for the lyophilized BPZE1 product did not affect the key molecular characteristics of the attenuated BPZE1 vaccine, i.e., the replacement of the B. pertussis ampG gene by that of E. coli, the deletion of the dnt gene, as assessed by specific PCRs, and the modifications of the PT S1 subunit gene that result in genetically inactivated PT, as assessed by a qPCR procedure, able to detect one putative reversion among 10⁶ genome equivalents.

While the RMC and Tg are generally indicative of the expected stability, there is no substitute for real time stability. Therefore, the lyophilized BPZE1 formulation was subjected to a real-time stability study at -20° C. ± 10° C., 5° C. ± 3° C. and 22.5° C. ± 2.5° C. The lyophilized BPZE1 formulation manufactured by direct dilution and by concentration & diafiltration, show that the formulation was stable when stored at -20° C. ± 10° C., 5° C. ± 3° C. and 22.5° C. ± 2.5° C. over a period of at least 24 months, as the CFU counts did not drop below the specification of 0.2 - 4 10⁹ CFU/ml during storage.

The adherence and colonization kinetics in the liquid and the lyophilized formulations were evaluated in mice using a liquid formulation stored at -70° C., containing 5% sucrose in PBS. The phase 1b clinical study showed that this liquid formulation led to colonization of >80% of the subjects, even though PBS is hypertonic as compared to the salinity of the respiratory tract. The decrease in salinity from PBS + 5% sucrose in the liquid formulation to the lower osmolarity of the Thijs medium + 10% sucrose did not affect the adherence or the colonization of the murine nasal cavity. The in-vivo colonization kinetics and protective potency were also assessed up to 24 months of storage at 3 different temperatures. Although 2-year storage at +5° C. ± 3° C. or +22.5° C. ± 2.5° C. appeared to slightly, but significantly reduce the adherence and the speed of colonization, this had only a minimal effect on vaccine potency, since the lyophilized BPZE1 formulation stored for 24 months at any of the temperatures tested still provided protection, i.e. a more than 100-fold reduction in bacterial load compared to non-vaccinated controls seven days after challenge.

Glass vials conduct heat/cold very poorly, since typically the contact of the bottom of the glass to the lyophilizer shelf is limited to 3 points. During freezing, this poor conduction leads to inhomogenous cooling of vials, i.e. the liquid in some vials will have initiated crystal formation while the liquid in others will remain liquid significantly longer. Especially in larger lyophilizers these inhomogenous heat/cold transfer issues can lead to relatively large differences in time between the first and the last vial to freeze.

It was hypothesized that the differences in duration from initiation of ice crystal formation to reaching the Tg′, i.e. the temperature at which water is no longer mobile, have an impact on bacterial survival after lyophilization is complete. Ice crystal formation means that the dissolved components of the lyophilization buffer increase in molarity, including the salts. The high salt concentration is likely to damage the outer membrane of B. pertussis, or any other bacterium, yeast, fungus or virus, so that duration of the phase in which high salt concentrations are present should be shortened if possible. Small scale research indicated that for the lyophilization buffer used, when cooled very slowly, ice crystal formation in the vials took place abruptly at -5.8° C., the crystallization point, with most vials showing abrupt crystal formation within a minute after each other, while usually ice crystal formation between the first and last vial can take an hour or more.

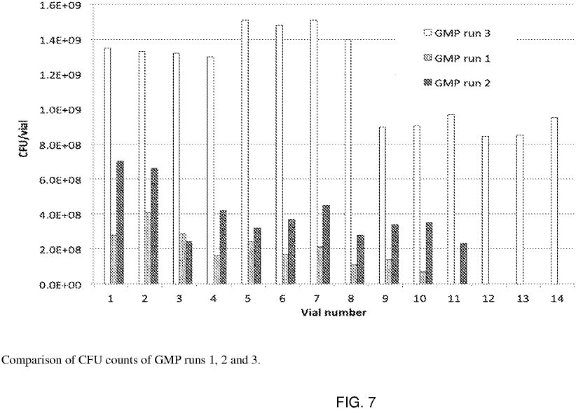

While the Tg′ is only reached at -34° C. for this formulation, initiation of crystal formation in the vials all at around the same time at -5.8° C. means that the homogeneity between the vials will increase since the starting point prior to crystallization is the same for all vials. As an example, in FIGS. 7, 3 runs (GMP run 1, 2 and 3) are compared, lyophilized in a production scale lyophilizer. The vials of GMP run 1 and 2 were cooled from ambient temperature to -50° C. using a ramp of 1° C./minute. The vials of GMP run 3 were cooled from ambient to -5° C. at a rate of 1° C./minute, at which the temperature was held for 1.5 hours, followed by freezing to -50° C., at a rate of 1° C./minute. The CFU counts of GMP run 1, 2 and 3 are compared in FIG. 7, showing a 3 to almost 6-fold difference between the highest and lowest CFU count for GMP run 2 and 1, respectively. For GMP run 3 the difference between the highest and lowest vial is less than 2-fold. Table 5 shows the same information, normalizing the highest CFU count for each batch to 100%.

TABLE 5

Comparison of CFU counts of GMP runs 1, 2 and 3 in tabulated format, normalizing the highest CFU count to 100% per batch.

| vial | GMP run 1 | GMP run 2 | GMP run 3 |
|---|---|---|---|
| 1 | 100 | 100 | 100 |
| 2 | 71 | 94 | 100 |
| 3 | 68 | 64 | 98 |
| 4 | 59 | 60 | 93 |
| 5 | 51 | 53 | 89 |
| 6 | 41 | 50 | 88 |
| 7 | 39 | 49 | 87 |
| 8 | 34 | 46 | 86 |
| 9 | 27 | 40 | 64 |
| 10 | 17 | 34 | 63 |
| 11 | | 33 | 60 |
| 12 | | | 59 |
| 13 | | | 57 |
| 14 | | | 56 |

The introduction of a pre-crystallization hold step prior to freezing to the Tg′ is as follows. Of a given lyophilization buffer the crystallization temperature is determined by slowly cooling the buffer and noting the temperature at which the onset of crystallization occurs. The pre-crystallization hold step can be defined as a hold step of half an hour to several hours, depending on the size of the lyophilizer, at 0. 1° C. to several degrees above the crystallization temperature, depending on the variability of the temperature of the cooling liquid running through the lyophilizer shelves.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of making a lyophilized vaccine comprising live attenuated Bordetella bacteria, the method comprising the steps of :
harvesting Bordetella bacteria from a culture at an OD₆₀₀ between 0.4 and 1.6;
mixing the harvested Bordetella bacteria with a lyophilization buffer comprising 5-65% by weight a cryoprotectant sugar and having a temperature between 2-35° C., wherein the ratio of harvested Bordetella bacteria to lyophilization buffer is between 5:1 and 1:5 by volume;
lyophilizing the mixture of the Bordetella bacteria and the lyophilization buffer ; wherein the hold time between the harvesting and lyophilization steps is less than 48 hours; and
collecting the lyophilized Bordetella bacteria.

2. The method of claim 1, wherein the Bordetella bacteria are a strain of Bordetella pertussis.

3. The method of claim 2, wherein the strain of Bordetella pertussis is a BPZE strain.

4. The method of claim 3, wherein the BPZE strain is BPZE1.

5. The method of claim 1, wherein the Bordetella bacteria from cultures at an OD₆₀₀ between 0.4 and 1.0.

6. The method of claim 1, wherein the Bordetella bacteria from cultures at an OD₆₀₀ less than 1.0.

7. The method of claim 1, wherein the cryoprotectant sugar is sucrose.

8. The method of claim 1, wherein the lyophilization buffer comprises a nutrient substrate.

9. The method of claim 8, wherein the nutrient substrate is glutamate.

10. The method of claim 1, wherein the hold time between the harvesting and lyophilization steps is less than 36 hours.

11. The method of claim 1, wherein the lyophilization step comprises a precrystallization hold step wherein the mixture of the Bordetella bacteria and the lyophilization buffer is held at 0.1 to 10° C. above the crystallization temperature of the mixture for 0.5-10 hours prior to further cooling.

12. The method of claim 1, further comprising a step of concentrating the harvested Bordetella bacteria to an OD₆₀₀ of 1.0 - 30.0 prior to the mixing step.

13. A lyophilized vaccine product comprising live attenuated Bordetella bacteria made according to a method comprising the steps of:
harvesting Bordetella bacteria from cultures at an OD₆₀₀ between 0.4 and 1.6;
concentrating the harvested Bordetella bacteria from cultures to an OD₆₀₀ of 1.0 - 30.0 ;
mixing the concentrated Bordetella bacteria with a lyophilization buffer comprising 5-65% by weight a cryoprotectant sugar and a temperature between 2 - 35° C., wherein the ratio of concentrated Bordetella bacteria to lyophilization buffer is between 5 :1 and 1 :5 by volume;
lyophilizing the mixture of the Bordetella bacteria and the lyophilization buffer, wherein the hold time between the harvesting and lyophilization steps is less than 48 hours; and
collecting the lyophilized Bordetella bacteria.

14. The lyophilized vaccine product of claim 13, wherein the product has a shelf life of at least two years when stored at 22.5° C., and at least 20% of the bacteria in the product remains viable after the lyophilization step.

15. The lyophilized vaccine product of claim 13, wherein, after the lyophilization step, the collected lyophilized bacteria are able to prevent or reduce infection of a subject’s respiratory tract with a pathogenic strain of Bordetella pertussis.
